(19) United States
(12) Patent Application Publication
(10) Pub. No.: US 2013/0177976 A1
(43) Pub. Date: Jul. 11, 2013

(54) COMBINATION OF CATIONIC POLYMERS AND POLYSACCHARIDES NANOPARTICLES AS A GENE CARRIER

(71) Applicant: Kaohsiung Medical University, Kaohsiung City (TW)

(72) Inventors: LI-FANG WANG, KAOHSIUNG CITY (TW); YU-LUN LO, NANTOU COUNTY (TW); YAO-SHENG WANG, KAOHSIUNG CITY (TW)

(73) Assignee: KAOHSIUNG MEDICAL UNIVERSITY, KAOHSIUNG CITY (TW)

(21) Appl. No.: 13/782,312

(22) Filed: Mar. 1, 2013

Related U.S. Application Data
(63) Continuation-in-part of application No. 13/299,826, filed on Nov. 18, 2011.

(30) Foreign Application Priority Data
Jul. 25, 2011 (TW) 100126253

(51) Int. Cl. C12N 15/85; C08F 22/10

(52) U.S. Cl. CPC C12N 15/85; C08F 22/10
USPC 435/320.1; 526/238.2

(57) ABSTRACT

The present invention discloses a gene carrier and the preparation method thereof. The chondroitin sulfate (CS) is reacted with methacrylic anhydride (MA) to form a chondroitin sulfate-methacrylate (CSMA), which is further covalently bound with 2-(dimethylamino)ethyl methacrylate (DMAEMA) to form the “CM-DM” gene carrier. The novel nonviral and lower cytotoxic gene delivery vector/carrier with the sugar functionality reduced the cytotoxicity of poly(2-dimethylaminoethyl methacrylate) (PDMAEMA), and can successfully deliver plasmid DNA to cancer cells via the caveolae-mediated and CD44-mediated endocytosis mechanisms.

### CROSS-REFERENCE TO RELATED APPLICATION AND CLAIM OF PRIORITY

This application is a continuation-in-part application of U.S. patent application Ser. No. 13/299,826 filed on Nov. 18, 2011, entitled “POLYSACCHARIDE-GRAFTED POLYETHYLENIMINE AS A GENE CARRIER”, and claims the benefits of the foreign priority application, i.e. TW 100126253 filed on Jul. 25, 2011, the contents of which are hereby incorporated by reference in its entirety.

### FIELD OF THE INVENTION

The present invention relates to a gene carrier. In particular, the present invention relates to a polysaccharide-grafted poly(2-dimethylaminoethyl methacrylate) as a gene carrier or a nonviral gene delivery vector.

### BACKGROUND OF THE INVENTION

Polyethylenimine (PEI) is one of the most potent gene carriers, and the high transfection efficiency of PEI has been postulated to relate to its buffering capacity, which leads to the accumulation of protons bought in by endosomal ATPase and an influx of chloride anions, triggering endosome swelling and disruption, followed by the release of gene drugs such as DNA or siRNA into cytoplasm.

The inventors of the present invention discloses a PAAIO-PEI nanoparticle prepared by using PEI and Fe₃O₄ as the materials in U.S. patent application Ser. No. 13/074,491 filed on Mar. 29, 2011. The PAAIO-PEI nanoparticle can be a non-viral gene carrier for carrying genetic material, is able to sustain superparamagnetic property, has less cytotoxicity than PEI, and shows better transgene expression efficiency than PEI under the disturbance of fetal bovine serum.

Additionally, Taiwan patent publication No. 201018523 discloses a liposome. For avoiding the activity attenuation of liposome-binding protein which is caused by chemical modification on liposome and reducing the purification steps of the chemically modified liposome, in that patent application, the positive charged polymers (e.g. PEI) and the surfactant polymers are distributed on the neutral lipid membrane with hollow spherical structure via non-covalently bonded combination, to form liposome. However, the patent publication does not overcome the cytotoxicity generated by PEI.

Although PEI has the above advantages and the applications in the prior art, for overcoming PEI's toxicity and owning it high capacity of passing through cellular membrane, the solution to PEI's high toxicity is still a problem necessary to be solved in the clinic application.

It is therefore attempted by the applicant to deal with the above situation encountered in the prior art.

### SUMMARY OF THE INVENTION

The natural negative charged polysaccharide, i.e. chondroitin sulfate (CS), is adopted as the material in the present invention, and CS is modified with covalent bonding, so that the double bond-modified CS is reacted with PEI via Michael addition to form the covalent bond in between, and a low toxicity and high water-soluble gene carrier is formed. In addition, the high capability nano-leveled gene carrier can be obtained by adjusting the ratio between CS and PEI to control the positive charge-negative charge ratio. The CS-modified PEI may differ a cellular internalization mechanism from PEI, resulting in the reduced cytotoxicity, but retains the gene transfecting efficiency as PEI does.

The present invention provides a preparation method of a gene carrier, including steps of: reacting CS with methacrylic anhydride (MA) to form chondroitin sulfate-methacrylate (CSMA); and covalently bonding PEI with CSMA to form the gene carrier.

After CSMA is generated, CSMA is precipitated with ethanol. Before PEI and CSMA are covalently bonded, PEI is mixed with pyridine. After the gene carrier is prepared, the gene carrier is dialyzed to remove the redundant PEI using the dialysis membrane. In addition, the gene carrier is capable of carrying the genetic material such as DNA, RNA, complementary DNA (cDNA), micro RNA, small interfering RNA and so on.

The present invention further provides a gene carrier prepared based on the above preparation method, the gene carrier includes CS, methacrylate bonded with CS, and PEI, wherein MA is covalently bonded with CS to form CSMA, and then PEI is covalently bonded with the methacrylate group of the CSMA.

Preferably, the prepared gene carrier is capable of carrying the genetic material described.

The present invention further provides a gene carrier, including: a saccharide (or carbohydrate) having a double-bond group; and a positive charge molecule having an amino group covalently bonded with the double-bond group.

The saccharide includes polysaccharides such as CS, chitosan, chitin, hyaluronic acid, dextran, and heparin. The double-bond group is originated from acrylate or methacrylate and is a carbon-carbon double-bond group such as acryloyl chloride, acrylic anhydride, methacryloyl chloride, and methacrylic anhydride. The positive charged molecule includes PEI and polylysine and other cationic polymers with primary amine groups.

The present invention further provides a preparation method of a gene carrier, including steps of: reacting CS with MA to form CSMA; and covalently copolymerizing 2-(dimethylamino)ethyl methacrylate (DMAEMA) with CSMA to form a gene carrier. Preferably, CSMA has a first methacrylate group, and DMAEMA has a second methacrylate group covalently copolymerized with the first methacrylate group. Preferably, the second methacrylate group can be originated from other monomers in addition to DMAEMA, including but not limited to 2-(diethylamino)ethyl methacrylate (DEAEMA), N-[3-(dimethylamino)propyl]methacrylamide (DMAPMA), 3-aminopropyl methacrylamide (APMA), 2-(diisopropylamino)ethyl methacrylate (DPAEMA), glycidyl methacrylate (GMA), (2-hydroxy-3-(2-aminoethyl)amino)propyl methacrylate (HAEAPMA), N-(2-hydroxypropyl)methacrylamide (HPMA), 2-lactobionamidoethyl methacrylamide (LAEMA), methacryloxysuccinimide (MAS), poly(ethylene glycol) methacrylate (PEGMA), 2-aminoethyl methacrylate (AMA), polyethylenimine methacrylate (PEI-MA), tert-butyl acrylate (tBA) and 2-hydroxyethyl methacrylate (HEMA).

The present invention further provides a gene carrier based on the above preparation method, including: the CS; the MA bonded with the CS to form CSMA; and the DMAEMA covalently copolymerized with the CSMA.

The present invention further provides a gene carrier, including: a saccharide having a first methacrylate group; and a monomer having a second methacrylate group covalently copolymerized with the first methacrylate group. Preferably, the saccharide is a polysaccharide. Preferably, the polysaccharide has functional groups capable of methacrylating and may be alginic acid, cellulose, chondroitin sulfate, chitosan, chitin, dextran, glycogen, heparin, hyaluronic acid, starch and so on.

The above objectives and advantages of the present invention will become more readily apparent to those ordinarily skilled in the art after reviewing the following detailed descriptions and accompanying drawings.

### BRIEF DESCRIPTION OF THE DRAWINGS

FIG. 1(a) to FIG. 1(c) respectively depict the ¹H-NMR spectra of (a) double bond-modified CSMA, (b) PEI and (c) CSMA-PEI.

FIG. 2 depicts the Fourier transform analysis spectra of CSMA, PEI and CSMA-PEI.

FIG. 3 depicts the UV/visible spectra of CSMA, PEI and CSMA-PEI.

FIG. 4 depicts the buffering capability analytic spectrum of CSMA, PEI and CSMA-PEI.

FIG. 5(a) to FIG. 5(c) respectively depicts the size comparison of CSMA-PEI/pEGFP-C1 nanoparticles which are stored in 10% FBS for (a) 0 hour, (b) 2 hours and (c) 4 hours after CSMA-PEI gene carriers with various amounts of PEI are mixed with pEGFP-C 1 with different N/P ratios.

FIG. 6 depicts the fluorescence comparison of CSMA-PEI/pEGFP-C1 nanoparticles after transfection, wherein the nanoparticles are prepared with various amounts of PEI and different N/P ratios.

FIG. 7 depicts the cytotoxicity of the various concentrations of CSMA-PEI against U87 cells.

FIG. 8 depicts the cytotoxicity of CSMA-PEI/pEGFP-C1 nanoparticles transfected into U87 cells.

FIG. 9 depicts the cytotoxicity of CSMA-PEI/pGL3 nanoparticles transfected into U87 cells.

FIG. 10 depicts the relative luminescence intensity of CSMA-PEI/pGL3 nanoparticles transfected into U87 cells.

FIGS. 11(a), 11(b) and 11(c) respectively depict the ¹H-NMR spectra of (a) CSMA, (b) PDMAEMA and (c) CM40-DM copolymer (each of 10 mg/mL in D₂O).

FIGS. 12(a) and 12(b) respectively depict (a) the FT-IR spectra and (b) the acid-base titration profiles of CSMA, PDMAEMA, CM25-DM and CM40-DM.

FIGS. 13(a) and 13(b) respectively depict the MTT assay of the cytotoxicity of PDMAEMA, CM25-DM and CM40-DM (a) at various concentrations (n=8) to HEK 293T cells, and (b) at various weight ratios (n=3) to HEK 293T cells (*p<0.01 and **p<0.001).

FIGS. 14(a), 14(b) and 14(c) respectively depict (a) the relative fluorescence intensity of pEGFP-C1 expression (n=3), (b) the luciferase activity of pGL3-Control expression normalized with protein amounts in the absence of 10% FBS (n=3) and (c) the luciferase activity of pGL3-Control expression normalized with protein amounts in the presence of 10% FBS (n=3), as the function of cationic polymers/pDNA weight ratio.

FIG. 15 depicts the transfection efficiency of pGL3 plasmid in HEK 293T cells (n=3, **p<0.001) after the cells had been treated with or without pinocytosis inhibitors.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

The gene carrier sought protected in the invention includes two parts, one is macromolecule such as hydrocarbon molecule (e.g. saccharide, polysaccharide and others), and the other is positive charge molecule. The amino group of the positive charge molecule forms a covalent bonding with the double-bonded group of hydrocarbon. The generated “hydrocarbon-positive charge molecule” is able to be a gene carrier, and further transfects genetic material such as DNA, RNA, complementary DNA (cDNA), microRNA, small interference RNA (siRNA) and so on into cells or tissues, so that the therapeutic or cytotoxic effect is achieved. Additionally, the examples of double-bond group are originated from molecules such as acrylate (CH₂═CHCOO⁻), methacrylate (CH₂═CMeCOO⁻) and others. In particular, the double-bond group is a carbon-carbon double bond group originated from molecules such as acryloyl chloride, acrylic anhydride, methacryloyl chloride, methacrylic anhydride and so on. The examples of positive charge molecule containing amino groups are chitosan, polyethylenimine (PEI), polylysine (or poly-L-lysine), protamine, poly(2-(dimethylamino)ethyl methacrylate) (DMAEMA), dendrimer and so on.

In one embodiment of the invention, chondroitin sulfate (CS) was modified with methacrylic anhydride (MA) to form chondroitin sulfate-methacrylate (CSMA), and then the positive charge polyethylenimine (PEI) formed covalent bond with CSMA to afford chondroitin sulfate-methacrylate-polyethylenimine (CSMA-PEI) gene carrier. This CSMA-PEI gene carrier was a positive charge water-soluble polymer, and a polyelectrolyte was formed to carry genetic material by using the electrostatic attraction force formed between the amino group and the genetic material, and the genetic material was delivered to cells or tissues via an endocytosis mechanism. The reaction formulas I and II of the embodiment of the invention were illustrated as follows.

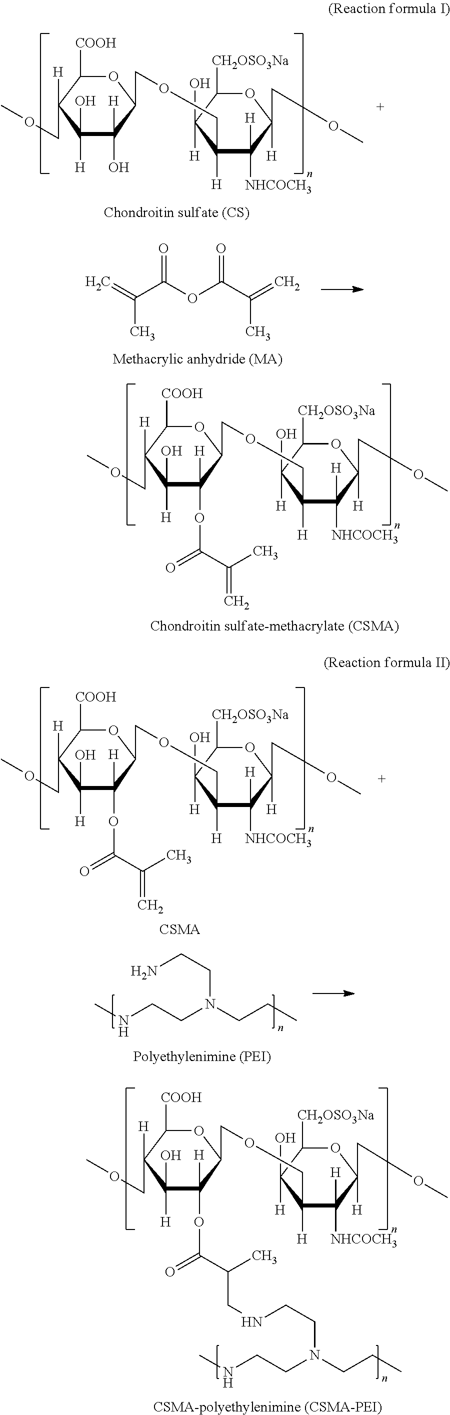

In addition to CS, other polysaccharides such as alginic acid, cellulose, chitosan, chitin, dextran, glycogen, heparin, starch, and hyaluronic acid also can be the candidates as the materials for preparing the gene carrier.

There is the second type of the gene carrier provided in the present invention, and such gene carrier also includes two parts, the saccharide having a methacrylate group and the DMAEMA monomer covalently copolymerized to form a copolymer. In another embodiment of the present invention, the methacrylate group of CSMA is covalently copolymerized with the methacrylate group of DMAEMA, and thus a gene carrier, illustrated as the following reaction formula III, is formed.

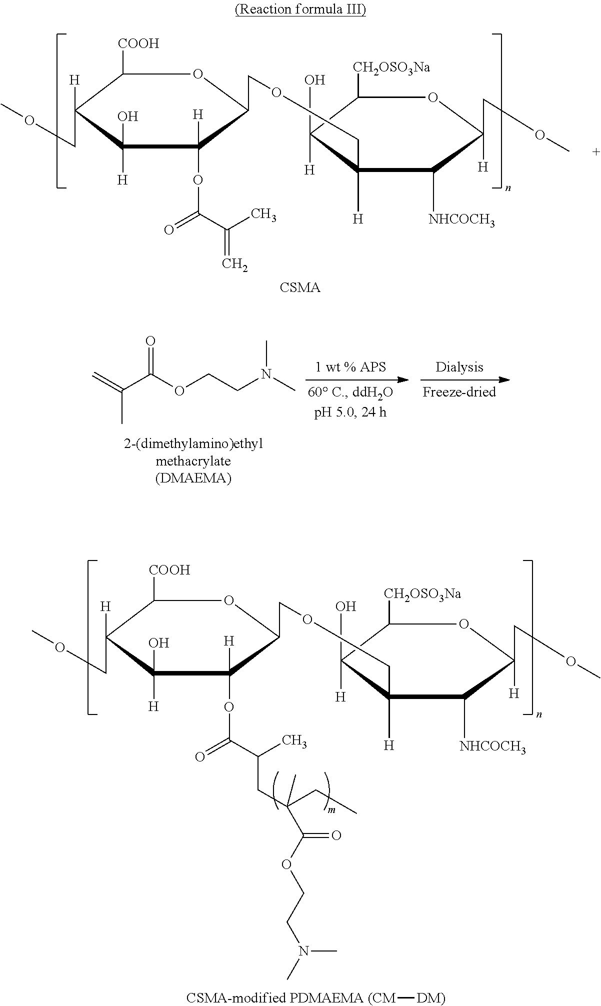

I. Preparation of chondroitin sulfate-methacrylate (CSMA):

Chondroitin sulfate (CS, 5 g) was dissolved in ddH₂O (250 mL), methacrylic anhydride (MA, 60 mL) was added dropwise into the CS solution, and then 5 N sodium hydroxide (NaOH) solution was added dropwise to react for 48 hours. Next, the reaction mixture was disposed at 4° C. overnight to cease the reaction. The reaction mixture was repeatedly precipitated with abundant ethanol and centrifuged (at 6,000 rpm for 5 minutes), and the precipitation was dried for 48 hours in the vacuum oven to obtain CSMA.

II. Preparation of CSMA-polyethylenimine (CSMA-PEI):

CSMA-PEI with different amounts of PEI could be prepared in accordance with the technology in the invention. CSMA-PEI with high, middle, or low amount of PEI defined as CP(H), CP(M), and CP(L) in the invention was illustrated as follows, and CSMA-PEI with other amount of PEI could be prepared in accordance with the similar method by the skilled person in this art.

PEI (1.5 g, molecular weight was about 10K Dalton) was dissolved in ddH₂O (1.5 L). After PEI was completely dissolved, pyridine (1.5 mL) was added and stirred for 2 hours. The CSMA solution (52.7 mg CSMA dissolved in 52.7 mL ddH₂O) was added dropwise into the aforementioned PEI solution and reacted in ambient temperature for 48 hours. The redundant PEI was removed using dialysis membrane (the allowable molecular weight to dialysis was about 25000 Dalton), the CSMA-PEI solution was lyophilized to obtain CSMA-PEI with high amount of PEI. CSMA-PEI with middle or low amount of PEI was prepared using the same preparation method and the PEI starting material (m.w. about 10K Dalton) of 300 mg and 100 mg, respectively.

III. Preparation and Properties of CSMA-PEI/pDNA Nanoparticles:

The preparation of CSMA-PEI gene carrier for carrying genetic materials was made by controlling the concentration of CSMA-PEI gene carrier to adjust the ratio of CSMA-PEI gene carrier to genetic material (such as plasmid DNA (pDNA)) in terms of N/P ratio. CSMA-PEI gene carrier and plasmid DNA solution with the same volumes were mixed at a ratio of 1 to 9 and then vortexed immediately. CSMA-PEI/pDNA nanoparticles were disposed at ambient temperature for 30 minutes to perform complete complex before analysis. N/P value can be an integral number such as 1, 2 to 9 or a non-integral number from 1 to 9, such as 1.5, 2.5 and so on. The used plasmid DNA in the experiments of the invention includes but not limit in the commercial pEGFP-C 1 (Clontech), pGL3 (Promega), other commercial plasmids or self-designed and prepared plasmids.

The conjugation ability of CSMA-PEI gene carrier to the genetic material could be evaluated by agarose gel electrophoresis. The CSMA-PEI/pDNA nanoparticles prepared based on the aforementioned method and different N/P ratios were used to determine the stability of nanoparticles using 0.8% agarose gel electrophoresis after mixing for different time periods in presence or absence of 10% fetal bovine serum (FBS).

Dynamic Light Scattering (DLS) and Zeta Potential.

The averaged hydrodynamic diameter and zeta potential of CSMA-PEI/pDNA were measured by laser Doppler anemometry using a Zetasizer Nano ZS instrument (Marlvern, Worcestershire, UK). Light scattering measurements were carried out with a laser at 633 nm with a 90° scattering angle. The concentration of the sample was 0.1 mg/mL and the temperature was maintained at 25° C. Polystyrene nanospheres (220±6 nm and −50 mV; Duke Scientific, USA) were used to verify the performance of the instrument. The particle size and zeta potential of each sample were performed in triplicate.

Transmission electron microscopy (TEM).

The size and morphology of magnetoplexes were visualized by cryo-TEM (Jeol JEM-1200, Tokyo, Japan). A carbon coated 200 mesh copper specimen grid (Agar Scientific Ltd. Essex, UK) was glow-discharged for 1.5 minutes. One drop of the sample solution was deposited on the grid and left to air-dry at room temperature, and was then examined with an electron microscope.

IV. Cytotoxicity Assay:

U87 cells (human glioblastoma cell line) were seeded in 12-well tissue culture plates at a density of 1×10⁵ cells per well in MEM (Minimum essential medium) containing 10% FBS for 24 hours. Cytotoxicity of CSMA-PEI nanoparticles was evaluated by determining the cell viability after 4 hours incubation of cells with CSMA-PEI gene carrier (or CSMA-PEI/pDNA nanoparticles) in a serum-free MEM followed by 72 hours post incubation in the MEM containing 10% FBS at the same condition. The number of viable cells and viability were determined by estimation of their mitochondrial reductase activity using the tetrazolium-based colorimetric method (MTT conversion test) known by the skilled person in this art.

V. Transfection Efficiency:

U87 cells were seeded at a density of 1×10⁵/well in 12 well plates and incubated in MEM (Minimum essential medium) containing 10% FBS for 24 hours before transfection. When the cells were at 50% to 70% confluence, the culture medium was replaced with 1 mL of MEM with or without 10% FBS. In addition, pEGFP-C1 (control, 4 μg) and CSMA-PEI/pEGFP-C1 nanoparticles (4 μg) were prepared, and the medium was replaced with fresh complete-medium and the cells were incubated for 48 hours post transfection after pEGFP-C 1 or CSMA-PEI/pEGFP-C 1 nanoparticles were cultured with cells for 6 hour incubation. The green fluorescent protein (GFP) expression was directly visualized under a fluorescence microscope.

For the luciferase assay, the procedures as stated above were made to determine the transfection efficiency of the CSMA-PEI/pGL3 nanoparticles compared with naked pGL3 plasmid DNA (as a negative control), Lipofectamine (a positive control), and PEI/pGL3 polyplex at a weight ratio of 10 (a positive control) in U87 cells. To quantify the luciferase expression, transfected cells were rinsed gently with 1 mL of 0.1 M PBS (phosphate buffered saline, twice), and then added to 200 μL/well of lysis buffer (0.1 M Tris-HCl, 2 mM ethylenediaminetetraacetic acid (EDTA), and 0.1% Triton X-100, pH 7.8) and let stand overnight at −20° C. Next day, each cell lysate was warmed to room temperature and centrifuged at high speed for 30 minutes. The luciferase activity was monitored using the TopCount NXT™ microplate scintillation and luminescence counter (Perkin Elmer, N.J., USA) after mixing the supernatant with the luciferase assay reagent (Promega, Madison, Wis., USA). The total protein content of the cell lysate was examined using a BCA protein assay kit (Pierce Rockford, Ill., USA).

VI. Characteristics of CSMA-PEI Gene Carrier:

Since PEI was known to has high transfection ability and was the most widely used non-viral carrier in clinics, its cytotoxicity was still high. Therefore, in the invention, PEI was bound to the double-bonded CSMA based on the transfection ability of PEI and covalent bonding, and the stable and positive charge CSMA-PEI gene carrier was formed by regulating the ratio of CSMA to PEI.

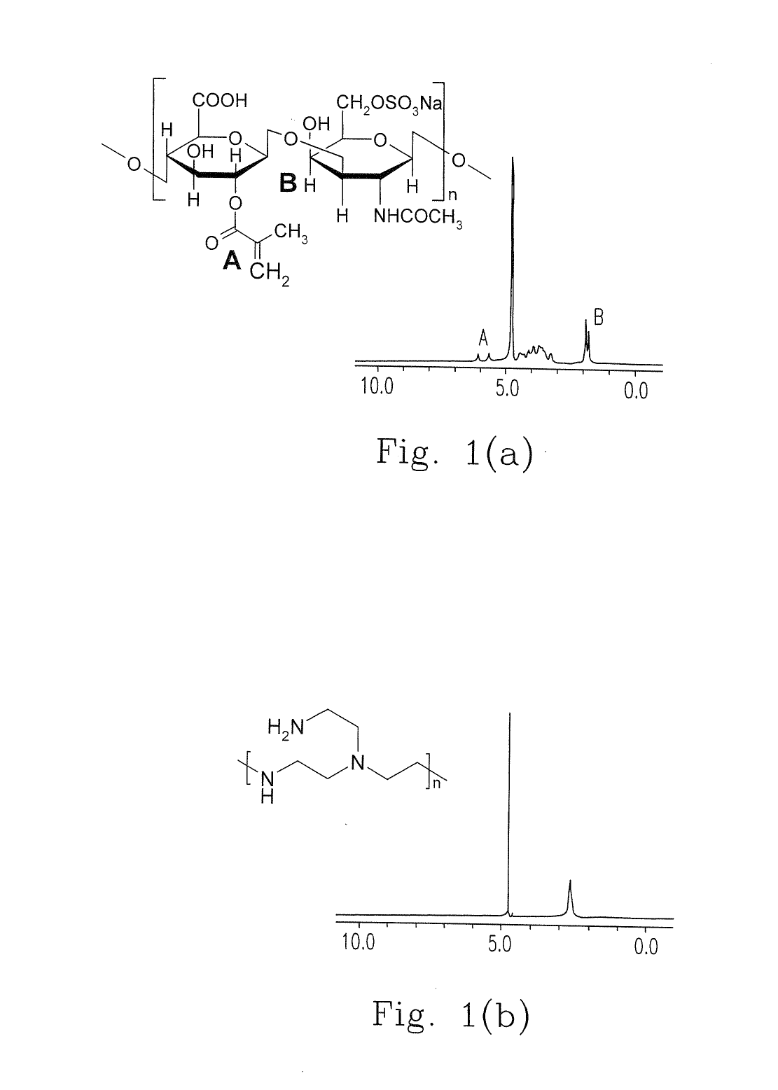

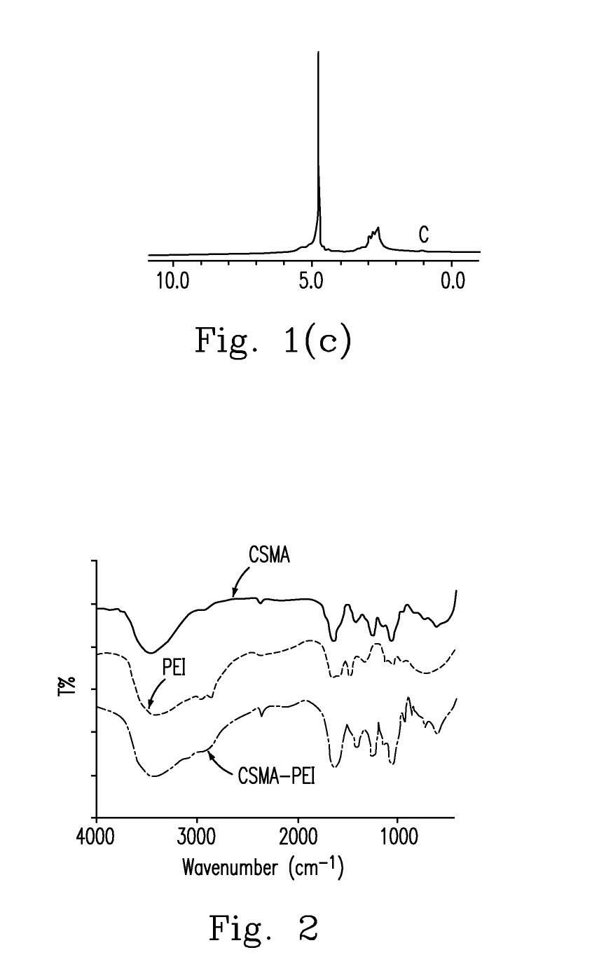

Please refer to FIGS. 1(a) to 1(c), respectively depict the ¹H-NMR spectra of (a) CSMA, (b) PEI and (c) CSMA-PEI. It could be known that Michael addition was performed between CSMA and PEI. The double-bond portion (i.e. the denotation “A”, d=5.6 ppm, 6.1 ppm) of CSMA in FIG. 1(a) was reacted with the amino group (d=2.6 ppm) of PEI in FIG. 1(b) to form carbon-nitrogen single bond, and ¹H-NMR spectrum of CSMA-PEI (i.e. the denotation “C” in FIG. 1(c)) showed the signal at d=1.1 ppm. Additionally, the denotation “B” in FIG. 1(b) was referred to CSMA. Furthermore, it was proved from DLS experiment that no particle was reproduced in hydrous solution (not shown), and thus CSMA and PEI formed the CSMA-PEI gene carrier by the covalent bond rather than attraction force of charge.

In quality, please refer to FIG. 2, which depicts the Fourier transform analysis spectra of CSMA, PEI, and CSMA-PEI. The analytic technology was well known by the skilled person in this art, and its experimental method was not illustrated herein. In FIG. 2, the peaks of CSMA-PEI showed the characteristic bonding of CSMA and PEI, and the more significant part was primary amine of PEI (2846 cm⁻¹, 1948 cm⁻¹). CSMA originally only had amide peak while the peak of primary amine appeared post Michael addition with PEI, proving that CSMA-PEI gene carrier was successfully synthesized in the invention.

In quantification, please refer to FIG. 3, which depicts the UV/visible spectra of CSMA, PEI, and CSMA-PEI. The spectrum analysis technology was well known by the skilled person in this art, and its experimental method was not illustrated herein. Since the lone-pair electrons of amino group of PEI and cupric bromide formed chelate, which had specific absorption at a wavelength of 630 nm, the amount of CSMA bound to PEI could be determined by using PEI standard curve. Since CSMA did not have specific absorption at 630 nm, it also could be known that covalent bonding was formed between CSMA and PEI.

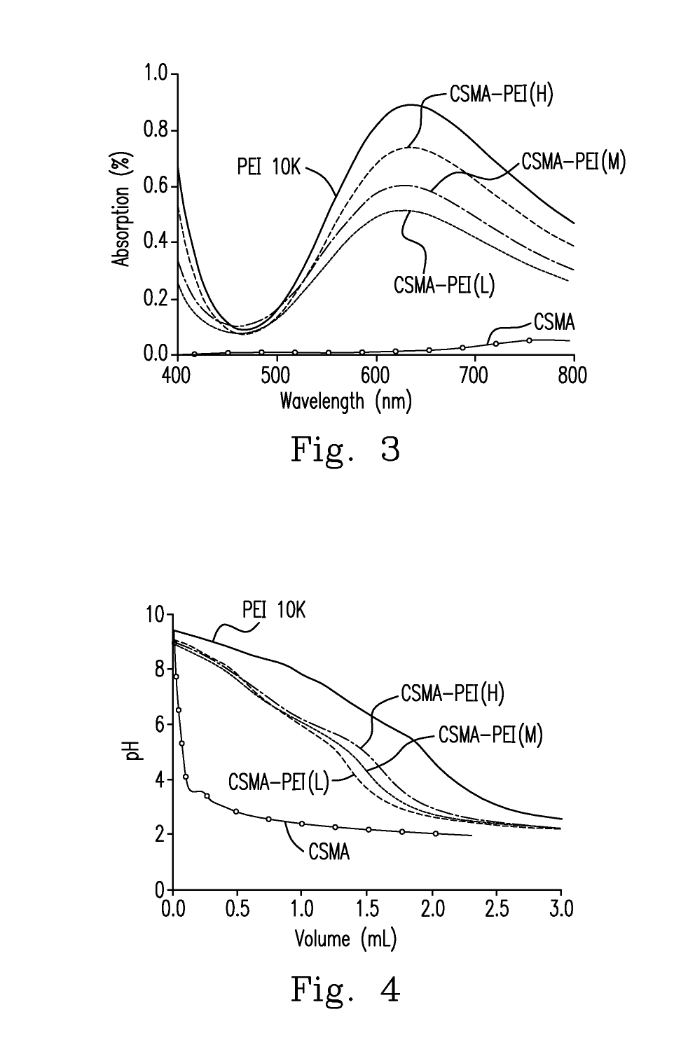

As the properties of PEI disclosed in Paragraph [0003] of the invention, the buffering capacity which is very important to gene carrier would be determined in this experiment. Therefore, buffering capacity assay of CSMA-PEI gene carrier was also determined in the invention, and the aqueous solution (material concentration of 1 mg/mL) was titrated with 0.1 N HCl to observe the variation of pH value. As shown in FIG. 4, CSMA-PEI of the respective ratios could sustain the more constant pH value, indicating that CSMA-PEI of the invention still maintained the buffering efficiency in cells.

In particle size determination, please refer to Table 1, which shows that the diameter of CSMA-PEI/pEGFP-C 1 nanoparticle was smaller than 185 nm after CSMA-PEI gene carrier with different amounts of PEI carried pEGFP-C1 plasmid DNA, and the size was gradually reduced with the increase in N/P ratio. From the surface potential analysis of CSMA-PEI/pEGPF-C1 nanoparticles, it would be known that nanoparticles of other N/P ratios carried positive charge except that surface potential was negative at N/P=1. It could also be known from TEM that CSMA-PEI/pEGPF-C1 nanoparticles showed as round shape (data not shown).

TABLE 1
DLS analysis of CSMA-PEI/pEGPF-C1 nanoparticles

| | Intensity of scattered | Polydispersity | | Zeta potential |
|---|---|---|---|---|
| | light (kcps) | index (PDI) | Size (nm) | (mV) |
| CSMA-PEI/pEGFP-CP(H) | | | | |
| N/P = 1 | 104.17 ± 5.24 | 0.353 ± 0.040 | 182.47 ± 10.66 | −24.10 ± 0.99 |
| N/P = 3 | 154.75 ± 4.60 | 0.366 ± 0.092 | 167.90 ± 1.13 | 9.66 ± 0.33 |
| N/P = 5 | 143.65 ± 11.24 | 0.326 ± 0.004 | 119.50 ± 4.38 | 17.90 ± 2.69 |
| N/P = 7 | 136.25 ± 10.68 | 0.539 ± 0.122 | 76.25 ± 5.31 | 20.00 ± 2.40 |
| CSMA-PEI/pEGFP-CP(M) | | | | |
| N/P = 1 | 104.05 ± 9.83 | 0.355 ± 0.024 | 142.20 ± 14.28 | −23.30 ± 1.13 |
| N/P = 3 | 179.20 ± 60.81 | 0.235 ± 0.043 | 128.40 ± 16.26 | 2.16 ± 2.81 |
| N/P = 5 | 209.00 ± 12.73 | 0.306 ± 0.038 | 133.95 ± 0.21 | 15.90 ± 1.48 |
| N/P = 7 | 190.00 ± 2.40 | 0.562 ± 0.074 | 87.23 ± 9.27 | 15.60 ± 1.84 |
| CSMA-PEI/pEGFP-CP(L) | | | | |
| N/P = 1 | 94.05 ± 5.73 | 0.689 ± 0.118 | 127.85 ± 18.03 | −18.60 ± 0.21 |
| N/P = 3 | 170.60 ± 5.80 | 0.516 ± 0.065 | 120.90 ± 7.78 | 19.20 ± 0.14 |
| N/P = 5 | 167.80 ± 14.00 | 0.543 ± 0.236 | 85.99 ± 0.11 | 16.20 ± 0.42 |
| N/P = 7 | 134.80 ± 31.68 | 0.696 ± 0.076 | 68.83 ± 11.84 | 22.80 ± 3.25 |

VII. Influence of Serum on CSMA-PEI/pEGPF-C1 Nanoparticles:

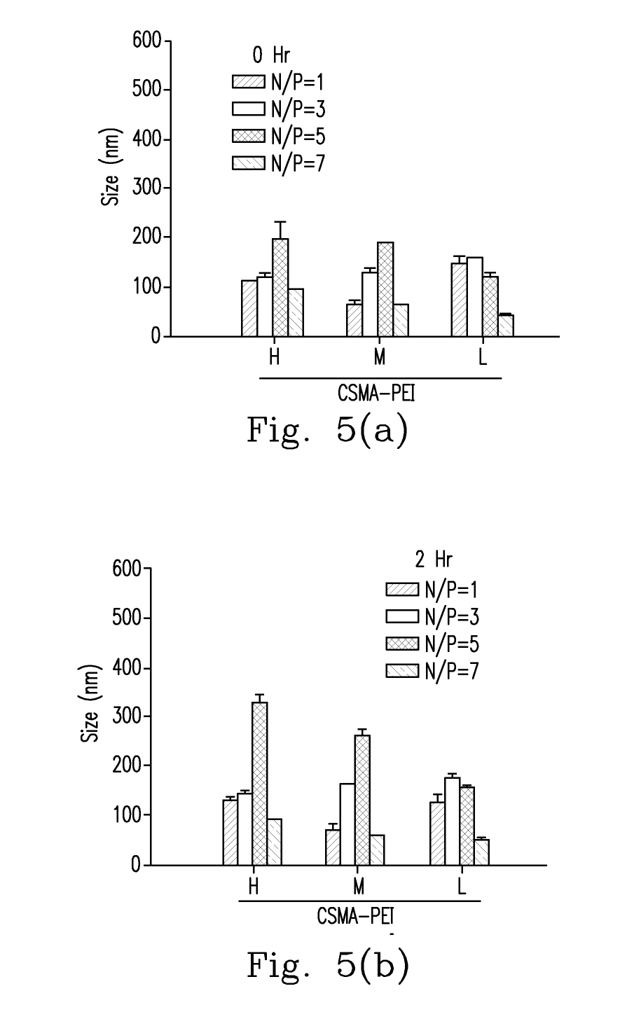

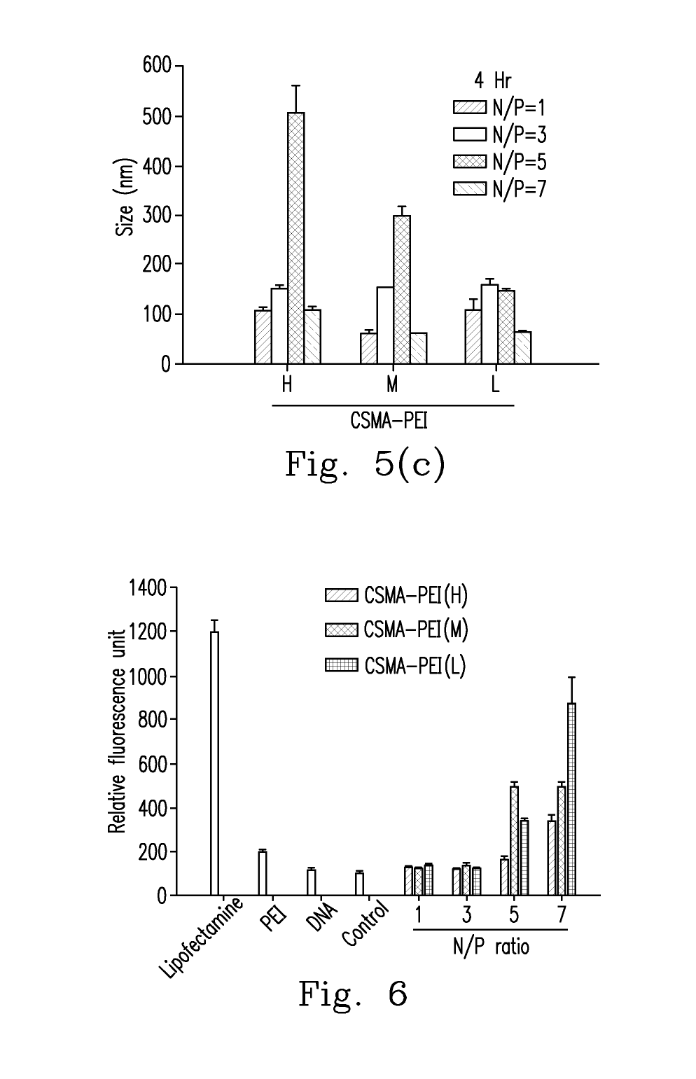

Please refer to FIGS. 5(a) to 5(c), the mixing of CSMA-PEI/pEGPF-C1 nanoparticles (based on the various N/P ratios) with 10% FBS would make nanoparticles influenced at different degrees subjected to serum, wherein N/P=7 had less influence and the diameter of nanoparticles sustained below 100 nm (compared with sizes in Table 1). As CSMA-PEI gene carriers with different amounts of PEI (i.e. CP(H), CP(M), and CP(L)), the size of CSMA-PEI/pEGPF-C1 nanoparticle formed by “CSMA-PEI” gene carrier and plasmid DNA had the lowest influence subjected to serum.

For understanding the protection ability of CSMA-PEI gene carrier of the invention on plasmid DNA, whether plasmid DNA was encapsulated by CSMA-PEI gene carrier was determined using agarose gel electrophoresis. Results showed that a partial of plasmid DNA still exposed from CSMA-PEI/pEGPF-C1 at N/P=1 while the results with respect to other N/P ratios showed that CSMA-PEI gene carrier completely encapsulated the plasmid DNA (data not shown). Additionally, CSMA-PEI/pEGPF-C1 nanoparticles was not influenced by serum under the condition of 10% FBS, and plasmid DNA was still exposed out at N/P=1 (data not shown), confirming that the gene carrier having polyelectrolyte structure in the invention had very high stability.

VIII. Efficiency of CSMA-PEI/pDNA Nanoparticles on Transfecting Genes:

In this experiment, the transfection efficiency of gene carrier carrying gene was evaluated by green fluorescence expression. Please refer to FIG. 6; it could be known that CSMA-PEI/pEGFP-C1 nanoparticles had high green fluorescence expression at high, middle and low PEI contents, and transfection increased with an increase in N/P ratio. The transfection effect of U87 cells was the best at N/P=7, and compared with “PEI”, the transfection efficiency of the CSMA-PEI gene carriers with high, middle, or low PEI content which carried pEGFP-C 1 increased.

It could be known from FIG. 6 that pEGFP-C1 was delivered into U87 cells by CSMA-PEI gene carrier to abundantly express green fluorescence protein and cells were not died, indicating that high biocompatibility of CS leads to reducing the toxicity of PEI and makes the transfection efficiency of CSMA-PEI gene carrier better than that of PEI.

IX. Cytotoxicity of CSMA-PEI Gene Carrier and CSMA-PEI/pDNA Nanoparticles:

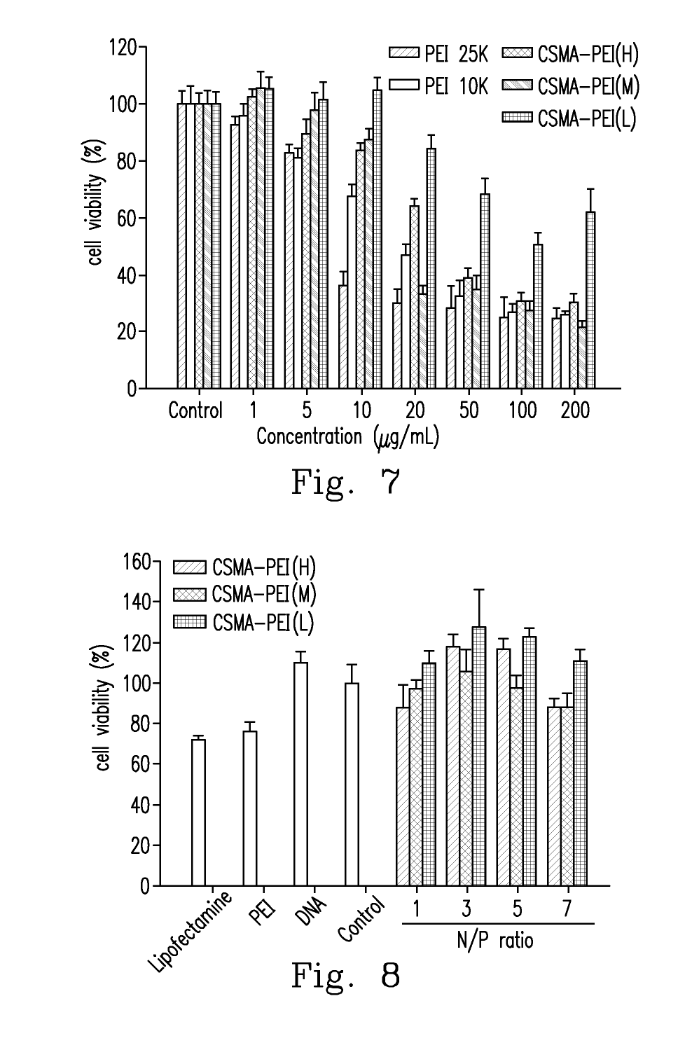

Please refer to FIG. 7; the cytotoxicity of the modified CSMA-PEI gene carrier was significantly reduced. CSMA-PEI gene carrier still sustained low toxicity compared with “PEI (control)” as a concentration of CSMA-PEI was 10 μg/mL, in particular in “CSMA-PEI (L)” group. When CSMA-PEI/pEGFP-C 1 nanoparticles were made at various N/P ratios, the viability of U87 cells was up to more than 90% as compared with other controls, indicating that CSMA-PEI gene carrier and CSMA-PEI/pEGFP-C 1 nanoparticles of the invention had low cytotoxicity (FIG. 8).

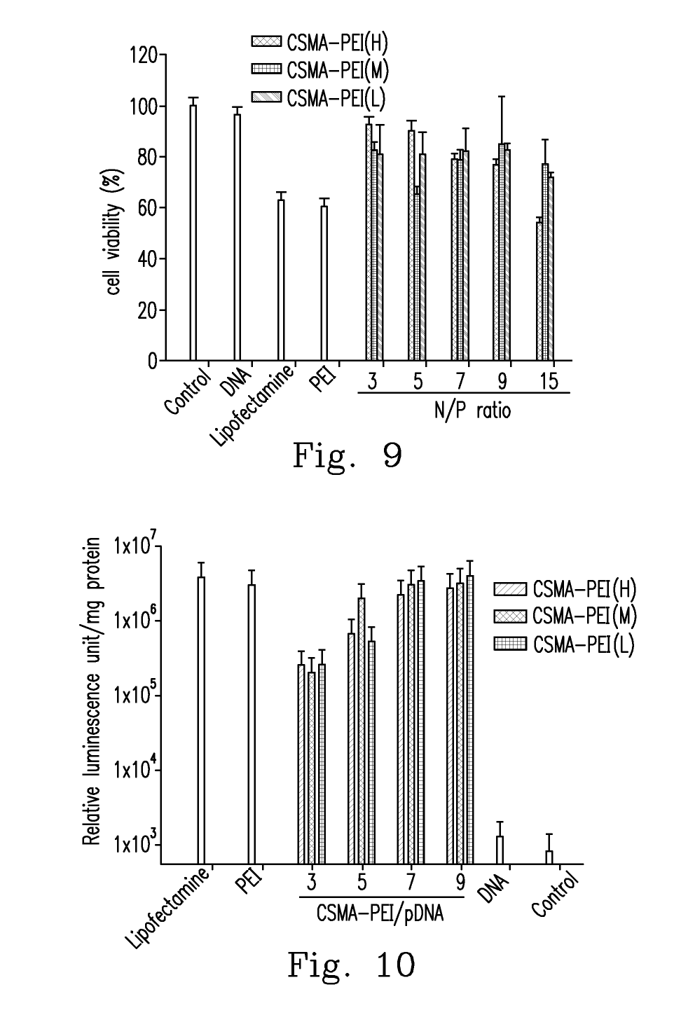

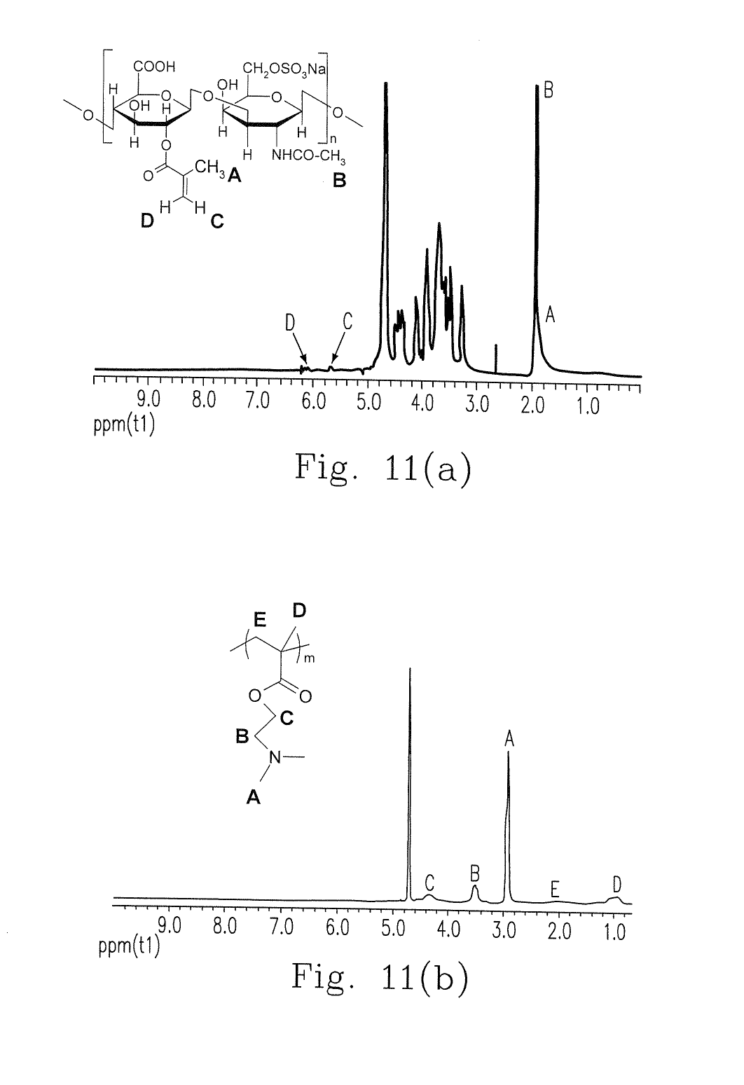

In addition to test the cytotoxicity of the gene carrier with the fluorescent pEGFP-C1, a luminescent plasmid pGL3 was also performed on cytotoxicity test of gene carrier with the pGL3. Please refer to FIG. 9; it was known that CSMA-PEI/pGL3 nanoparticles had the minimized cytotoxicity and compatible transfection efficiency (FIG. 10) as compared with control groups, “PEI/pGL3 at N/P=10” group and “Lipofectamine/pGL3” group. Thus, CSMA-PEI gene carrier was an adequate material to transport a genetic material into cells with the significantly reduced cytotoxicity (FIG. 8).

X. Preparation and Characteristics of CSMA-Modified PDMAEMA (CM-DM):

The synthesis route of the CM-DM gene carrier was illustrated as the above reaction formula III and was described in detail as follows. In the first set of experiments, CSMA to poly(2-dimethylaminoethyl methacrylate (PDMAEMA) was fixed at 25 weight percent (wt %) and the degrees of methacrylation in CSMA was verified from 5% to 70% to prepare the CM-DM gene carrier (or named as CM-DM copolymer). Next, the methacrylation substitution of CSMA was fixed at 5% and the CSMA of 5 wt % to 50 wt % with a 5% increment in feed was varied. The reaction concentration was controlled at 2.5 mg/mL in ddH₂O and the reaction mixture was adjusted to pH 5 using the acidic solution (e.g. 1 N HCl), followed by adding the free radical initiator (e.g. 1 wt % ammonium peroxodisulfate (APS)) relative to the total amount of CSMA and DMAEMA. The reaction mixture was degassed by at least one freeze-pump-thaw cycle, preferably three cycles. The reaction was carried out under argon atmosphere at 60° C. for 24 hours. The resultant solution (containing the CM-DM gene carrier) was purified by dialysis using a dialysis membrane (Spectra/Por MWCO 3,500; Spectrum Laboratories, Rancho Dominguez, Calif.) against ddH₂O. The final product was lyophilized.

In the first set of experiments, the CM-DM copolymers formed a gel type when the methacrylation was >5%, whereas in the second set, the CM-DM copolymers formed a gel when the CSMA feed percent was >40%. A numeral behind CM indicates the feed percent of CSMA in preparation of CM-DM. For example, CM25-DM and CM40-DM indicate the CSMA feed was controlled at 25% and 40% respectively. The CM-DM copolymers with a low content of CSMA showed similar cytotoxicity to PDMAEMA itself. Thus, CM25-DM and CM40-DM were selected for detailed characterizations.

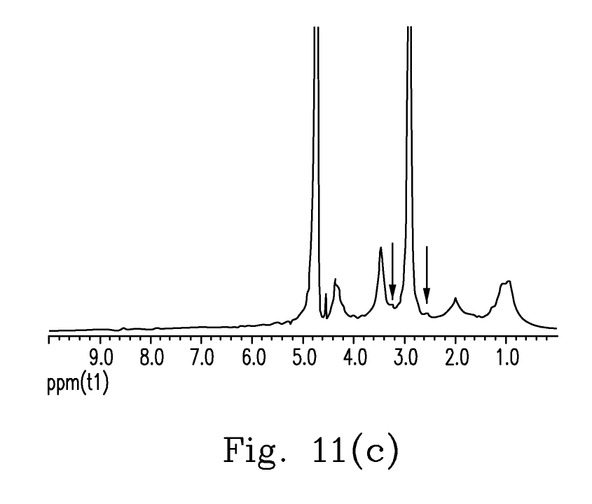

¹H-NMR experiments were recorded using a Varian Mercury plus-200 (200 MHz) NMR spectrometer in D₂O at a concentration of 10 mg/mL Please refer to FIGS. 11(a), 11(b) and 11(c), which respectively show the ¹H-NMR spectra of (a) CSMA, (b) PDMAEMA and (c) CM40-DM copolymer. Besides the characteristic peaks of PDMAEMA appearing at 1.0, 2.0, 2.9, 3.5 and 4.3 ppm (sequentially denoted as “D”, “E”, “A”, “B” and “C” in FIG. 11(b)), the CM40-DM also had the characteristic peaks of CSMA (indicated by arrows in FIG. 11(c)). Two protons of double bonds on CSMA at δ 5.6 and 6.1 ppm (the denotations “D” and “C” respectively in FIG. 11(a)) disappeared after the reaction among double bonds of CSMA and DMAEMA. The signal intensity at ˜2.0 ppm (the denotation “B” in FIG. 11(a)) manifested after CSMA had reacted with DMAEMA. This is because of the signal overlapping between three protons of acetamide on CSMA (indicated by the denotation “A” in FIG. 11(a)) and two protons on PDMAEMA (indicated by the denotation “E” in FIG. 11(b)).

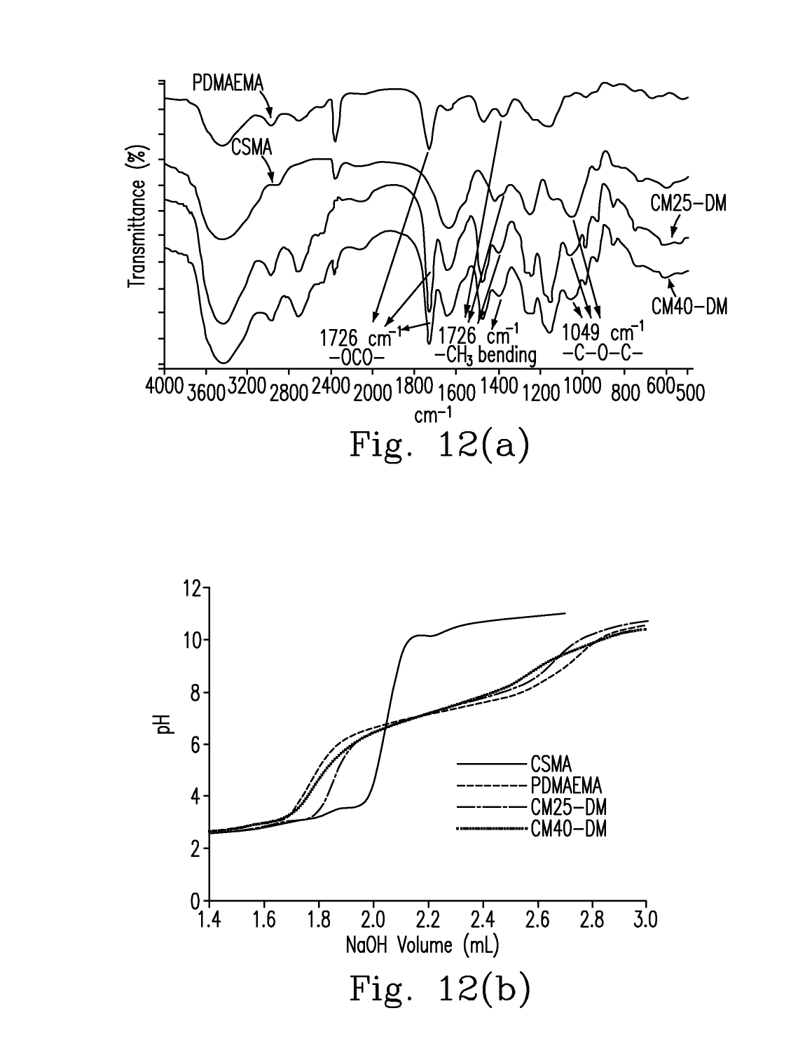

Furthermore, Fourier-transform infrared (FT-IR) spectra were acquired using a System 2000 spectrophotometer (Perkin-Elmer, Fremont, Calif.), and 64 scans were signal-averaged (range: 4000-400 cm⁻¹) at a resolution of 4 cm⁻¹. Please refer to FIG. 12(a), the FT-IR spectra of CM25-DM and CM40-DM showed the inherited peaks of CSMA and PDMAEMA, where the characteristic ester bonds on PDMAEMA peaked at 1726 cm⁻¹, and stretching bands on CSMA at 1646 cm⁻¹ (Amide I), 1350 cm⁻¹ (S=0) and 1049 cm⁻¹ (ether bond). The copolymer compositions of CM25-DM and CM40-DM were determined by FT-IR measurements. The intensity ratio of the —C—O—C— ether bond on CSMA at 1049 cm⁻¹ and the —COO— ester bond on PDMAEMA at 1726 cm⁻¹ were measured and plotted to generate a calibration curve using five known weight ratios of CSMA and PDMAEMA. The experimental weight ratios of CSMA in CM25-DM and CM40-DM were 24% and 38%, which were close to the initial feed ratios.

The molecular weight and molecular weight distribution of PDMAEMA and CM-DM were measured by gel permeation chromatography (GPC) using an Aglient 1100 series equipped with PL aquagel-OH 40 and PL aquagel-OH 60 columns, and a phosphate buffer solution (pH 7.4) was used as an eluent at a flow rate of 1 mL/min. Six monodisperse polyethylene glycol (PEG) standards were used to generate a calibration curve. The number-averaged molecular weights of PDMAEMA, CSMA, CM25-DM and CM40-DM were 1.03×10⁵, 1.09×10⁵, 1.12×10⁵ and 1.25×10⁵ g/mol with the PDI of 1.19, 1.21, 1.25, and 1.21, respectively based on the GPC diagrams (data not shown).

Acid-base titration was done using a PC-controlled system assembled with a 702 SM Titroprocessor, a 728 stirrer, and a PT-100 combination pH electrode (Metrohm, Herisau, Switzerland). To keep the ionic strength constant throughout the titration and for complete dissolution and protonation before titration, each 20-mg sample was added to 150 mM NaCl (20 mL), and the pH was adjusted to 2.0 using 0.1 N HCl. The titration was done by adding 0.1 N NaOH at 25.0±0.1° C. under an atmosphere of CO₂-free N₂. Please refer to FIG. 12(b), which depicts the acid/base titration profiles of CSMA, PDMAEMA, CM25-DM and CM40-DM. The CSMA did not show any buffering capacity, while CM25-DM and CM40-DM had a similar buffer capacity. The apparent pKa values of amino groups were 7.36±0.13, 7.31±0.28 and 6.91±0.24 for PDMAEMA, CM25-DM and CM40-DM, respectively, and these values were close to the value for PDMAEMA, 7.0 to 7.5.

XI. Preparation and Characteristics of PDMAEMA/pDNA and CM-DM/pDNA Polyplexes:

Each cationic polymer (PDMAEMA, CM25-DM and CM40-DM) was dissolved in ddH₂O to a final concentration of 1 mg/mL at pH 6 to pursue the protonated amino groups of PDMAEMA. The pDNA concentration was fixed at 3 μg/100 μL in ddH₂O to measure DNA binding and 4 μg/500 μL for other measurements. Equal volumes of PDMAEMA or CM-DM, and pDNA solutions were mixed and immediately vortexed at high speed for 60 seconds. At pH 6.0, the three cationic polymers and pDNA (e.g. pEGFP-C1 and pGL3) should be ionized to form polyelectrolyte complexes. The pDNA was well-complexed at a weight ratio>1.5 between cationic polymers and pDNA. The modification of CSMA with PDMAEMA to form CM-DM did not affect the binding ability with pDNA.

Additionally, the hydrodynamic diameters and zeta potentials of PDMAEMA/pDNA and CM-DM/pDNA polyplexes at various weight ratios were tabulated in Table 2. The particle diameters of polyplexes remarkably decreased when the weight ratio of cationic polymers/pDNA was prepared at w/w=2.4. For PDMAEMA/pDNA, the hydrodynamic diameter was 100 nm at w/w=1.5 and reduced to 46 nm at w/w=2.4. The CM25-DM/pDNA and CM40-DM/pDNA polyplexes showed a similar trend in decrease in the hydrodynamic diameter with an increase in a weight ratio from 1.5 to 2.4 (from 130 nm to 88 nm for CM25-DM/pDNA and from 116 nm to 86 nm for CM40-DM/pDNA). The hydrodynamic diameters increased at a weight ratio of w/w=5.8, i.e. 92 nm for CM25-DM/pDNA and 117 nm for CM40-DM/pDNA.

TABLE 2
Hydrodynamic diameters and zeta potentials at various weight ratio
of polymer/pDNA polyplexes in ddH₂O at pH 6.0*

| Polyplex | w/w | PDI | Dₕ (nm) | Zeta (mV) |
|---|---|---|---|---|
| PDMAEMA | 0.5 | 0.35 ± 0.03 | 150.8 ± 16.3 | −20.6 ± 0.1 |
| | 1.5 | 0.29 ± 0.09 | 100.5 ± 20 | 6.4 ± 0.6 |
| | 2.4 | 0.34 ± 0.02 | 46.5 ± 5.9 | 12.2 ± 5.3 |
| | 3.4 | 0.32 ± 0.01 | 42.7 ± 0.1 | 20.6 ± 0.1 |
| | 4.4 | 0.32 ± 0.01 | 47.8 ± 1.1 | 21.6 ± 0.7 |
| | 5.8 | 0.36 ± 0.01 | 49.9 ± 9.1 | 24.9 ± 16.9 |
| CM25-DM | 0.5 | 0.26 ± 0.05 | 100.6 ± 2.2 | −13.4 ± 0.1 |
| | 1.5 | 0.27 ± 0.04 | 130.5 ± 8.3 | 3.8 ± 2.4 |
| | 2.4 | 0.21 ± 0.01 | 88.5 ± 0.7 | 3.9 ± 1.0 |
| | 3.4 | 0.34 ± 0.03 | 76.7 ± 11.1 | 9.8 ± 0.1 |
| | 4.4 | 0.32 ± 0.08 | 74.3 ± 7.9 | 11.2 ± 0.1 |
| | 5.8 | 0.31 ± 0.09 | 91.7 ± 8.5 | 17.2 ± 0.1 |
| CM40-DM | 0.5 | 0.35 ± 0.03 | 96.3 ± 2.9 | −11.9 ± 1.3 |
| | 1.5 | 0.35 ± 0.03 | 115.8 ± 41.2 | 4.1 ± 0.3 |
| | 2.4 | 0.35 ± 0.03 | 86.3 ± 15.7 | 7.1 ± 0.9 |
| | 3.4 | 0.35 ± 0.03 | 81.2 ± 17.2 | 8.5 ± 0.7 |
| | 4.4 | 0.35 ± 0.03 | 91.5 ± 14.5 | 12.0 ± 0.8 |
| | 5.8 | 0.35 ± 0.03 | 117.5 ± 24.3 | 18.7 ± 0.8 |

*The concentration of sample is 0.1 mg/mL. The data are means ± SD of an experiment done in triplicate.

At the same weight ratio, the smaller hydrodynamic diameters of PDMAEMA/pDNA than those of CM-DM/pDNA were always found because of stronger electrostatic interactions in between. All three polyplexes showed a negative zeta-potential value at a weight ratio of 0.5 and turned to a positive value when the weight ratio was adjusted to >1.5. When comparing the zeta-potentials among three polyplexes at the same weight ratio, the PDMAEMA/pDNA polyplex always showed the highest value because of the absence of the negatively charged CSMA, which neutralized the positively charged PDMAEMA.

The hydrodynamic diameters of CM25-DM and CM40-DM in different pH values were tested because CM-DM is a zwitterions copolymer. Table 3 shows the hydrodynamic diameters and zeta-potentials of CM40-DM gene carrier in different pH values ranging from 5 to 8. The hydrodynamic diameters were within 99 to 136 nm with a broad PDI of 0.50 to 0.77 and zeta-potentials within 21 to 29 mV. For CM25-DM, both hydrodynamic diameter and zeta potential were immeasurable. This result implied CM25-DM was a sol-type polymer and CM40-DM approximately reached a critical point between a sol and a gel type.

TABLE 3
Hydrodynamic diameters and zeta potentials of CM40-DM in
different pH values*

| pH | Kcp | PDI | Dₕ (nm) | Zeta (mV) |
|---|---|---|---|---|
| 5 | 410 | 0.50 ± 0.09 | 99.4 ± 22.1 | 20.6 ± 5.8 |
| 6 | 381 | 0.62 ± 0.19 | 135.6 ± 47.7 | 22.6 ± 2.3 |
| 7 | 374 | 0.77 ± 0.21 | 106.7 ± 60.7 | 29.3 ± 1.1 |
| 8 | 566 | 0.56 ± 0.06 | 132.2 ± 29.3 | 25.1 ± 2.8 |

*The concentration of sample is 0.1 mg/mL. The data are means ± SD of an experiment done in triplicate.

The CM40-DM/pDNA polyplex at a weight ratio of 3.4 was used to determine the effect of different pH values on particle diameters and zeta-potentials at two time points-immediately after the polyplex preparation or after it had been left to stand for 30 minutes. In Table 4, the particles of CM40-DM/pDNA were stable at a pH range of 5 to 7 and slightly increased with an increase in pH as well as the time standing in a solution. At pH 8, the particle diameter remarkably increased after it had been left to stand for 30 minutes. The hydrodynamic diameter increased 3-fold from 119 nm to 351 nm and the zeta potential turned to a negative value.

TABLE 4
Hydrodynamic diameters and zeta potentials of CM40-DM/pDNA
polyplex at a weight ratio of 3.4 in different pH values*

| pH | Time (minutes) | Kcp | PDI | Dₕ (nm) | Zeta (mV) |
|---|---|---|---|---|---|
| 5 | 0 | 310 | 0.34 ± 0.18 | 41.4 ± 15.8 | 12.6 ± 3.5 |
| | 30 | 151 | 0.25 ± 0.04 | 46.1 ± 10.0 | 21.1 ± 6.5 |
| 6 | 0 | 610 | 0.38 ± 0.08 | 42.4 ± 4.8 | 8.6 ± 1.8 |
| | 30 | 296 | 0.35 ± 0.09 | 48.1 ± 8.0 | 14.2 ± 7.4 |
| 7 | 0 | 124 | 0.24 ± 0.01 | 73.0 ± 2.9 | 7.67 ± 0.4 |
| | 30 | 174 | 0.25 ± 0.05 | 88.2 ± 5.7 | 7.23 ± 0.6 |
| 8 | 0 | 186 | 0.32 ± 0.02 | 119.4 ± 8.4 | 7.5 ± 1.5 |
| | 30 | 287 | 0.51 ± 0.06 | 351.2 ± 79.0 | −0.4 ± 0.7 |

*The concentration of sample is 0.1 mg/mL. The data are means ± SD of an experiment done in triplicate.

The particle diameters prepared at a weight ratio of 3.4 and at pH 6.0 were 95.4±17.6 nm, 89.8±8.3 nm and 86.6±9.2 nm for PDMAEMA/pDNA, CM25-DM/pDNA and CM40-DM/pDNA, respectively, under the TEM scope. Furthermore, the DMAEMA/pDNA appeared to be assembled by many dots under the TEM scope because PDMAEMA and pDMA primarily formed complexation, followed by hierarchical association of multiple nanocomplexes into a cluster. In contrast, CM25-DM/pDNA or CM40-DM/pDNA formed spheroids with a smooth surface. A pH response in particle size of CM40-DM/pDNA at the weight ratio of 3.4 was also examined by TEM. The stable particles with an appreciable core and shell structure were clearly observed at pH of 5 to 7 but deformed at pH 8. At pH 8, the deprotonation of PDMAEMA resulted in reducing the electrostatic interaction forces with the negatively charged pDNA.

XII. Cytotoxicity Assay:

HEK 293T cells (human embryonic kidney 293T cell line) were seeded in 96-well tissue culture plates at a density of 5×10³/well in 100 μL DMEM containing 10% FBS. The cytotoxicity of PDMAEMA and CM-DM was evaluated by determining the cell viability after 24 hours of incubation with various concentrations of polymers (2.5 to 100 μg of polymer/mL) at 37° C., and then the cytotoxicities of copolymers/pDNA were examined at various weight ratios for 72 hours. The number of viable cells was determined by the MTT conversion test.

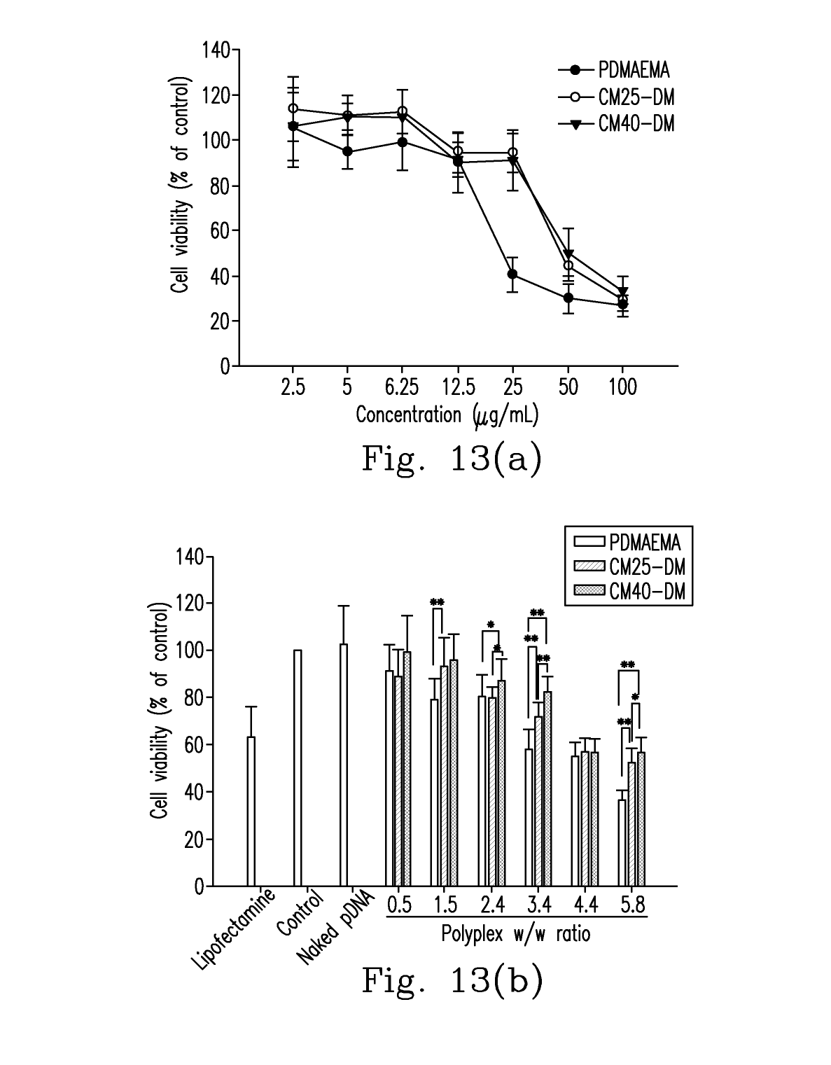

The cytotoxicity of PDMAEMA dramatically increased at a concentration ≧12.5 μg/mL The cell viability was ˜90% at 12.5 μg/mL and ˜40% at 25.0 μg/mL However, CM25-DM and CM40-DM remained 90% viable at 25.0 μg/mL After the introduction of CSMA to PDMAEMA, the cytotoxic issue of PDMAEMA is significantly improved (FIG. 13(a)). The dosages required inhibiting cell proliferation by 50% (IC₅₀) of PDMAEMA, CM25-DM and CM40-DM were 18.40±1.56 μg/mL, 35.88±6.08 μg/mL and 38.67±12.87 μg/mL, respectively. At the polymer/pDNA of w/w=1.5, the cell viability of CM25-DM/pDNA was higher than that of PDMAEMA/pDNA (p<0.001). At w/w=3.4, the cell viabilities were 60%, 73% and 84% and at w/w=5.8, they were 38%, 54% and 58%, for PDMAEMA/pDNA, CM25-DM/pDNA and CM40-DM/pDNA, respectively. Comparing cell viabilities with PDMAEMA/pDNA, the CM-DM/pDNA polyplexes had a significant improvement in cytotoxicity especially when a high polymer weight ratio was used (FIG. 13(b)).

XIII. In Vitro Transgene Expression:

This transfection assay was performed similar to the above “V. Transfection efficiency”. HEK 293T cells were seeded in 12 well plates (1×10⁵ cells/well) and incubated in DMEM containing 10% FBS for 24 hours before transfection. When the cells were at 50-70% confluence, the culture medium was replaced with DMEM (1 mL) with or without 10% FBS. Polyplexes with w/w of 0.5 to 5.8 were prepared using different amounts of polymers and a fixed pDNA amount (4 μg) to a final volume (500 μL). After 4 hour incubation, the medium were replaced with fresh complete-medium (1 mL), and the cells were incubated for 72 hour post-transfection. The GFP expression was directly visualized using the fluorescence microscope.

For the luciferase assay, the procedures stated above were repeated to determine the transfection efficiency in HEK 293T cells. The transfection efficiency of CM-DM/pGL3 and PDMAEMA/pGL3 polyplexes was compared with naked pDNA (a negative control) and Lipofectamine (a positive control). Other procedures were followed based on Paragraph [0053].

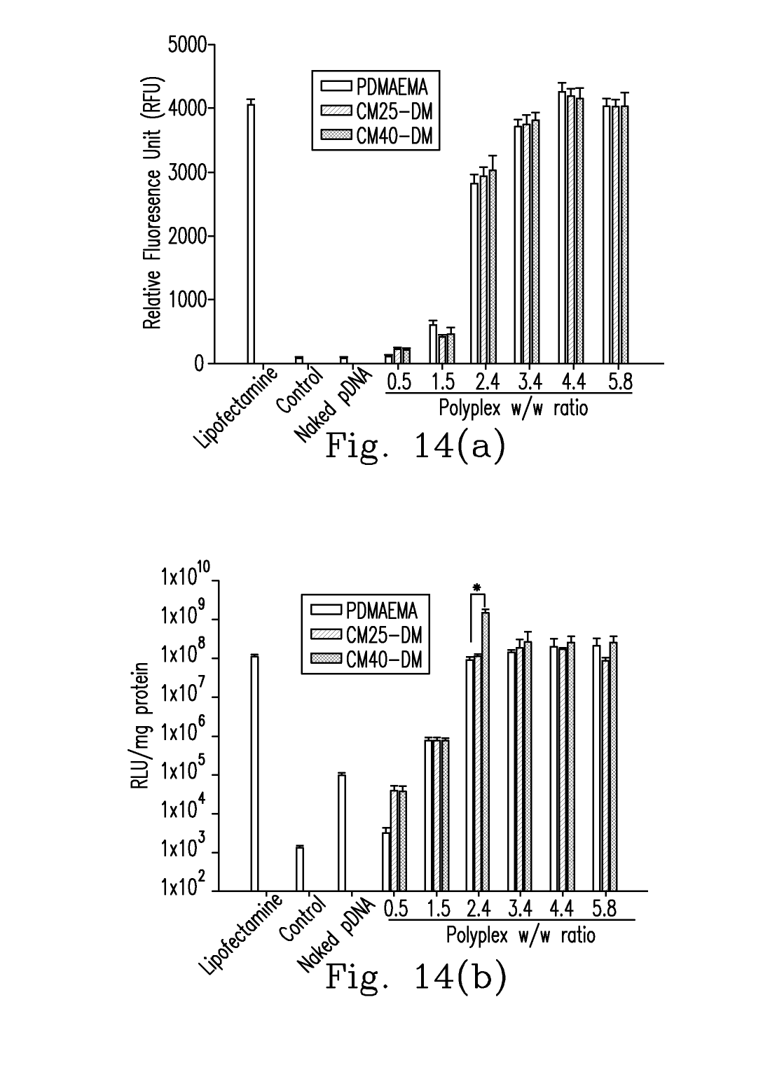

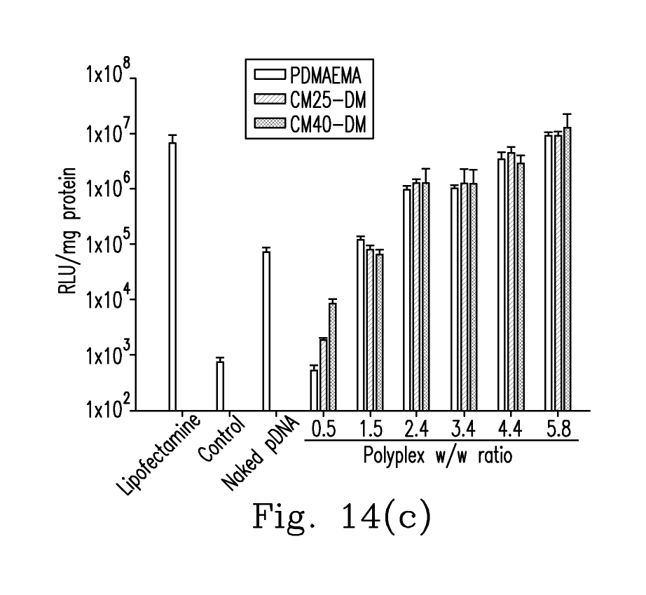

The green fluorescence expression was clearly observed when the weight ratio of polymers/pEGFP was ≧2.4 (data not shown). Please refer to FIG. 14(a); the relative fluorescence intensities among three polyplexes at w/w ≧3.4 were equal and comparable to that of Lipofectamine. Please refer to FIG. 14(b); the quantitative transfection ability of the polyplexes was measured and compared with that of Lipofectamine/pGL3. Among three polyplexes, the transfection efficiency of CM40-DM/pDNA increased at w/w=2.4 but was comparable at the other weight ratios. Please refer to FIG. 14(c); the transfection efficiency of each polyplex was approximately one order lower than itself without FBS. This fact was because of the competition between FBS and pDNA with cationic polymers, which reduced the pDNA concentration in the polyplexes, or because the FBS adsorption on the polyplex surface inhibits transgene expression. Either in the presence or absence of 10% FBS, there was no significant difference in transfection efficiency among the three test polyplexes.

XIV. Cellular Internalization:

To visualize the cellular internalization of CM-DM/pDNA polyplexes into cells, the CM-DM gene carrier was labeled with fluorescein isothiocyanate (FITC). In brief, the CM-DM gene carrier (100 mg) was dissolved in DMSO (10 mL), and FITC (10 mg) was added into the above solution in the presence of dilaurate dibutyltin (15 μL) and reacted at 60° C. for 3 hours. The reaction solution was prevented from the light and kept reacting at room temperature for 5 days. The reaction solution was precipitated into alcohol in an ice-bath, and the precipitate was separated by centrifugation followed by washing with alcohol till no FITC residue was detected in the alcohol solution using a fluorescence spectrophotometer (Cary Eclipse, Varian, Calif.). A sample concentration was adjusted to 0.1 mg/mL in ddH₂O. The maximum emission intensity of the sample at 520 nm was measured using an excitation wavelength of 490 nm. The FITC content of the sample was calculated based on an FITC calibration curve in a concentration range of 0 to 1.6 μg/mL in a mixture of methanol and ddH₂O (1/1 v/v).

Confocal Laser Scanning Microscope (CLSM).

The intracellular delivery of pDNA was observed using CLSM. HEK 293T cells were seeded at a density of 1.5×10⁵/well in 12 well plates containing one glass coverslip/well in DMEM supplemented with 10% FBS and then incubated for 24 hours. The polyplex was prepared at a weight ratio of 3.4 of CM40-DM and Cy5-labeled pGL3-control plasmid. The pGL3-control plasmid was labeled with Cy5 using a nucleic acid labeling kit (Minis Labelit Cy5 Labeling Kit: MIR-3700; Fisher Scientific Company, Pittsburgh, Pa.) following the manufacture's protocol. The cells were exposed to the polyplex for various time periods at 37° C. After they had been incubated, the medium containing the polyplex was removed. The coverslips were washed gently with 0.1 M PBS (1 mL) at pH 7.4 and then treated with Hoechest (1 mL/well) at 37° C. for 30 minutes. Next, the coverslips were removed, washed gently 5 times with 0.1 M PBS (1 mL), placed in a new empty well, and treated with 1 mL of 3.7% paraformaldehyde in 0.1 M PBS for 30 minutes to fix the cells. The cells on the coverslips were washed 3 times with PBS and mounted with fluorescent mounting medium on glass slides. A CLSM (Fv 1000; Olympus, Tokyo, Japan) was used for cell imaging.

CD44 Receptor Expression in U87 Cells and HEK 293T Cells.

An FITC conjugate anti-CD44 antibody against CD44 receptor assay was used to measure the expression of CD44 receptor in U87 and 293T cells. The cells were trypsinized and then washed in PBS buffer. The cells (3×10⁵) were then resuspended in PBS buffer and incubated with anti-human/mouse CD44 FITC antibody for 30 minutes on ice. The CD44 antibody concentration was based on the manufacturer's instructions. After the incubation, the cells were washed three times with PBS, resuspended in PBS buffer (1 mL), and then analyzed using a flow cytometer.

Three major internalization mechanisms of CM-DM/pDNA polyplexes were tested using their corresponding chemical inhibitors: chlorpromazine (10 μg/mL) for clathrin-mediated endocytosis, genistein (200 nM) and methyl-β-cyclodextrin (mβ-CD, 1 mM) for caveolae-mediated endocytosis, and wortmannin (50 nM) for macropinocytosis. These inhibitors were nontoxic to HEK 293T cells at the concentrations used. The flow cytometric diagram clearly shifted left when the cells (seeding density: 2×10⁵/well in a 6 well plate) were pretreated with genistein and mβ-CD, and remained intact when the cells were pretreated with chlorpromazine and wortmannin before the cells were treated with the FITC-conjugated CM40-DM/pDNA polyplex at a weight ratio of 3.4.

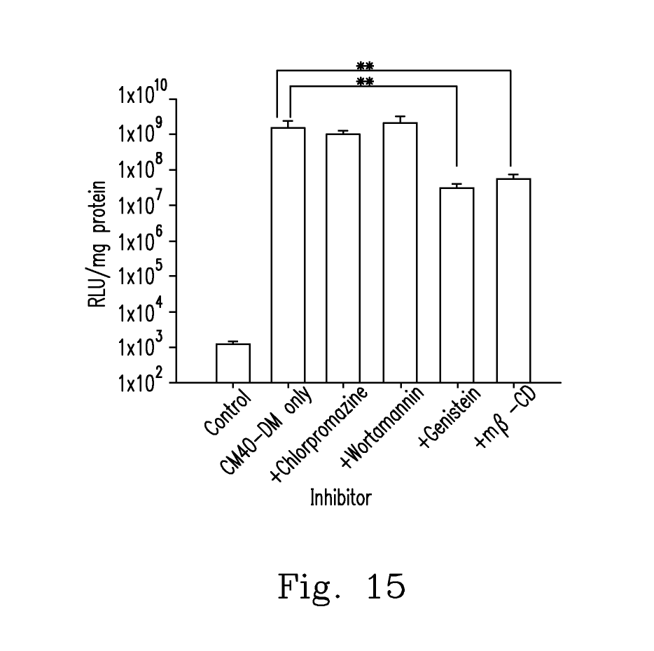

Since CSMA is internalized in cells via CD44 receptor-mediated endocytosis, the cellular uptake of CM40-DM/pDNA by this mechanism was tested. The FITC-linked CD44 antibody was used to clarify whether HEK 293T cells overexpress CD44 receptor. As compared with the control, no shift in the fluorescence intensity demonstrates low expression of the CD44 receptor in HEK 293T cells, while the remarkable increase in the fluorescence intensity demonstrates high expression of the CD44 receptor in U87 cells. The HEK 293T cells were pretreated with CSMA to block CD44 before exposure to CM40-DM/pDNA to reconfirm if the cells express the CD44 receptor. Because of low CD44 expression in HEK 293T cells, the cellular uptake of CM40-DM/pDNA was apparently not inhibited. In contrast, the CD44-overexpressed U87 cells were tested. The flow cytometric diagram clearly shifted left when the cells were pretreated with genistein, slightly shifted left when the cells were pretreated with CSMA, and remained intact when the cells were pretreated with chlorpromazine and wortmannin when the cells had been treated with the FITC-linked CM40-DM/pDNA polyplex. Please refer to FIG. 15; the luciferase gene expression of this polyplex in HEK 293T cells significantly decreased in the presence of genistein and mβ-CD (p <0.001).

The cellular internalization process of the polyplex was followed using CLSM at three time points. The nuclei were stained with Hoechst 33342 in blue. Intracellular uptake of pDNA in red increased with an increasing incubation time. At 2 hour, the pDNA release from the polyplex was clearly observed and the perinuclear region of the cells was reached. At 4 hour, the overlay of FITC-labeled CM40-DM and Cy5-labeled pDNA in orange was observed around the nuclei. The enhanced red fluorescence intensity indicated more pDNA molecules were released and entered the nuclei with a longer incubation time.

In conclusion, a novel nonviral gene delivery vector/carrier based on CSMA-modified PDMAEMA is provided in the present invention. The optimized conditions of the degree of methacrylation on CSMA (<5%) and the weight percent of CSMA (<40 wt %) to produce the sol-type CM-DM were determined. The introduction of CSMA to PDMAEMA reduced the intrinsic cytotoxicity of PDMAEMA itself. The CM-DM copolymers successfully delivered plasmid DNA to cancer cells, including but not limited in HEK 293T cells. CM-DM/pDNA had transfection efficiency close to PDMAEMA/pDNA and comparable to Lipofectamine/pDNA. The caveolae-mediated as well as CD44-mediated endocytosis mechanisms were involved in the cellular uptake of the CM-DM polyplexes. The CM-DM copolymers should be an excellent nonviral gene delivery vector than did PDMAEMA because of the lower cytotoxicity and having sugar functionality for multiple uses.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A preparation method of a gene carrier, comprising steps of:
reacting a chondroitin sulfate with a methacrylic anhydride to form a chondroitin sulfate-methacrylate; and
covalently copolymerizing a 2-(dimethylamino)ethyl methacrylate with the chondroitin sulfate-methacrylate to form the gene carrier.

2. The preparation method according to claim 1, wherein the 2-(dimethylamino)ethyl methacrylate and the chondroitin sulfate-methacrylate forms a reaction mixture and have a total amount before forming the gene carrier, and the preparation method further comprises steps of:
adjusting the reaction mixture to pH 5 to 8 by using an acidic solution; and
adding a free radical initiator relative to the total amount to the reaction mixture.

3. The preparation method according to claim 2, wherein the acidic solution is a hydrochloride solution, and the free radical initiator is 1 weight percent of an ammonium peroxodisulfate.

4. The preparation method according to claim 2 further comprising a step of degassing the reaction mixture by at least one freeze-pump-thaw cycle.

5. The preparation method according to claim 4, wherein the degassing step is carried out under an argon atmosphere at 60° C. for 24 hours to form a resultant solution containing the gene carrier.

6. The preparation method according to claim 5 further comprising a step of dialyzing the resultant solution to remove at least one of the redundant 2-(dimethylamino)ethyl methacrylate and the redundant chondroitin sulfate-methacrylate.

7. The preparation method according to claim 1 further comprising a step of precipitating the chondroitin sulfate-methacrylate with an ethanol.

8. The preparation method according to claim 1 further comprising a step of mixing the gene carrier with a genetic material.

9. The preparation method according to claim 8, wherein the genetic material is selected from the group consisting of a DNA, an RNA, a complementary DNA, a micro RNA, and a small interfering RNA.

10. The preparation method according to claim 1, wherein the chondroitin sulfate-methacrylate has a first methacrylate group, and the 2-(dimethylamino)ethyl methacrylate has a second methacrylate group covalently copolymerized with the first methacrylate group.

11. A gene carrier, comprising:
a chondroitin sulfate;
a methacrylic anhydride bonded with the chondroitin sulfate to form a chondroitin sulfate-methacrylate; and
a 2-(dimethylamino)ethyl methacrylate covalently copolymerized with a chondroitin sulfate-methacrylate to form the gene carrier.

12. The gene carrier according to claim 11, wherein the gene carrier is configured to carry a genetic material.

13. The preparation method according to claim 12, wherein the genetic material is selected from the group consisting of a DNA, an RNA, a complementary DNA, a micro RNA, and a small interfering RNA.

14. The gene carrier according to claim 11, wherein the chondroitin sulfate-methacrylate has a first methacrylate group containing a vinyl group, the 2-(dimethylamino)ethyl methacrylate has a second methacrylate group covalently copolymerized with the vinyl group.

15. A gene carrier, comprising:
a saccharide having a first methacrylate group; and
a monomer having a second methacrylate group covalently copolymerized with the first methacrylate group.

16. The gene carrier according to claim 15, wherein the saccharide further is a polysaccharide.

17. The gene carrier according to claim 16, wherein the polysaccharide has functional groups capable of methacrylating and is selected from the group consisting of alginic acid, cellulose, chondroitin sulfate, chitosan, chitin, dextran, glycogen, heparin, hyaluronic acid and starch.

18. The gene carrier according to claim 15, wherein the monomer is selected from the group consisting of 2-(dimethylamino)ethyl methacrylate (DMAEMA), 2-(diethylamino)ethyl methacrylate (DEAEMA), N-[3-(dimethylamino)propyl]methacrylamide (DMAPMA), 3-aminopropyl methacrylamide (APMA), 2-(diisopropylamino)ethyl methacrylate (DPAEMA), glycidyl methacrylate (GMA), (2-hydroxy-3-(2-aminoethyl)amino)propyl methacrylate (HAEAPMA), N-(2-hydroxypropyl)methacrylamide (HPMA), 2-lactobionamidoethyl methacrylamide (LAEMA), methacryloxysuccinimide (MAS), poly(ethylene glycol) methacrylate (PEGMA), 2-aminoethyl methacrylate (AMA), polyethylenimine methacrylate (PEI-MA), tert-butyl acrylate (tBA) and 2-hydroxyethyl methacrylate (HEMA).
